(19) United States
(12) Patent Application Publication
(10) Pub. No.: US 2025/0032665 A1
(43) Pub. Date: Jan. 30, 2025

(54) DYNAMIC COVALENT ADHESIVES: MOISTURE-ACTIVATED INSTANTANEOUS ADHESION

(71) Applicants: THE TRUSTEES OF THE UNIVERSITY OF PENNSYLVANIA, Philadelphia, PA (US); THE CHILDREN'S HOSPITAL OF PHILADELPHIA, Philadelphia, PA (US); LEHIGH UNIVERSITY, Bethlehem, PA (US)

(72) Inventors: Shu YANG, Blue Bell, PA (US); Mingtao CHEN, Zhejiang (CN); Alexander TUCKER, Philadelphia, PA (US); Anand JAGOTA, Bethlehem, PA (US); Yue WU, Bethlehem, PA (US)

(21) Appl. No.: 18/714,148

(22) PCT Filed: Nov. 29, 2022
(86) PCT No.: PCT/US2022/051155

§ 371 Date: May 29, 2024

Related U.S. Application Data
(60) Provisional application No. 63/283,622, filed on Nov. 29, 2021.

(51) Int. Cl. A61L 24/06

(52) U.S. Cl. CPC A61L 24/06

(57) ABSTRACT

A reversibly adhesive film, comprising: at least two hydroxyl-bearing polymer chains being crosslinked by one or more boronic ester bonds, the film having accessible hydroxyl groups at a surface of the film. An reversible adhesive, comprising: a first film layer, the first film layer comprising at least two first hydroxyl-bearing polymer chains crosslinked by first crosslinks that comprise one or more boronic ester bonds; and a second film layer, the first second layer comprising at least two second hydroxyl-bearing polymer chains crosslinked by second crosslinks that comprise one or more boronic ester bonds, wherein (1) the first hydroxyl-bearing polymer chains differ from the second hydroxyl-bearing polymer chains in one or more of composition and concentration, or (2) the boronic ester bonds are present in the first film layer at a different density than the boronic ester bonds in the second film layer, or (3) both (1) and (2).

### CROSS-REFERENCE TO RELATED APPLICATIONS

The present application claims priority to and the benefit of U.S. patent application No. 63/283,622, “Dynamic Covalent Adhesives: Moisture-Activated Instantaneous Adhesion” (filed Nov. 29, 2021), the entirety of which application is incorporated herein by reference for any and all purposes.

### TECHNICAL FIELD

The present disclosure relates to the field of reversible adhesive materials.

### BACKGROUND

Adhesion bonding at interfaces falls into two categories: 1) strong but slow and irreversible covalent bonds and 2) fast, reversible but weak non-covalent bonds. Synergizing the advantages from both categories remains challenging but pivotal for next-generation wound adhesives. Accordingly, there is a long-felt need in the art for adhesives that exhibit strong bond strength but can also be reversed.

### SUMMARY

Realizing fast and strong adhesion with effortless removal is challenging, especially for wound adhesives where the adherend surface is soft and/or hairy. We introduced dynamic covalent bonded matrices as adhesives to orchestrate the advantages from both covalent bond (strong adhesion) and dynamic bond (fast and reversible) while overcoming their respective limitations. Upon activation with water within seconds, the hydrated and soft surface ensures good conformal contact with the adherends and allows fast binding at the interfaces. The bulk adhesive film remains dry and is responsible for efficient load transmission. We demonstrate the potential of dynamic covalent bonds to address the trade-off between adhesion strength and adhesion time/reversibility.

The present disclosure first provides reversibly adhesive films, comprising: at least two hydroxyl-bearing polymer chains, the at least two hydroxyl-bearing polymer chains being crosslinked by crosslinks that comprise one or more boronic ester bonds, the film having accessible hydroxyl groups at a surface of the film, and at least a portion of the film being in a dried state.

Also provided are methods, comprising contacting a hydrated portion of a film according to the present disclosure (e.g., any one of Aspects 1-17) to an adherend for a time sufficient to give rise to adhesion between the film and the adherend.

Further provided are methods, comprising hydrating a film according to the present disclosure (e.g., any one of Aspects 1-17) that is adhered to an adherend so as to reduce adhesion between the film and the adherend.

Also disclosed are reversible adhesives, comprising: a first film layer, the first film layer comprising at least two first hydroxyl-bearing polymer chains crosslinked by first crosslinks that comprise one or more boronic ester bonds; and a second film layer, the first second layer comprising at least two second hydroxyl-bearing polymer chains crosslinked by second crosslinks that comprise one or more boronic ester bonds, wherein (1) the first hydroxyl-bearing polymer chains differ from the second hydroxyl-bearing polymer chains in one or more of composition and concentration, or (2) the boronic ester bonds are present in the first film layer at a different density than the boronic ester bonds in the second film layer, or (3) both (1) and (2).

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. Schematic illustration of PVA/BA adhesives: (a) Instant surface softening upon water activation allows (b) fast adhering to wound site through dynamic covalent bonds.

FIG. 2. Characterization of PVA/BA aqueous solution: (a) Physical appearance of PVA/BA with different crosslinking density and (b) corresponding schematic demonstration; (c) Shear sweep of PVA/BA 5/1, 8/1, 15/1, 30/1, and 50/1 solution (20 w % in DI water); Frequency sweep of (d) PVA/BA 5/1, (e) 8/1, and (f) 15/1 aqueous solution (20 w % in DI water). Shear storage modulus (G′) with increasing frequency (filled blue circles), shear storage modulus (G′) with decreasing frequency (filled red circles), shear loss modulus (G″) with increasing frequency (empty blue diamonds), shear loss modulus (G″) with increasing frequency (empty red diamonds).

FIG. 3. Adhesion performance of PVA/BA adhesives on glasses. In (a) single-joint lap shear tests, (b) peak adhesions of PVA/BA 5/1, 15/1, 30/1, 50/1 films are compared with commercial product Dermabond® with (c) representative curves. Similarly, in (d) tensile tests, (e) peak adhesion forces (after 2 minutes of adhering) of PVA/BA 5/1, 15/1, 30/1, 50/1 films after water activation (10 seconds) are compared with commercial product Tegaderm® with (f) representative curves. PVA/BA adhesive films were activated with water for 10 seconds and adhered to substrate at 37° C. for 2 minutes.

FIG. 4. Adhesion performance of PVA/BA adhesives on hairy mouse skins. In (a) tensile tests, (b) peak adhesions of PVA/BA 5/1, 15/1, 30/1, 50/1 films are compared with commercial product Dermabond® with (c) representative curves. Similarly, in (d) incision tests, (e) peak adhesion forces of PVA/BA 5/1, 15/1, 30/1, 50/1 films are compared with commercial product Dermabond® with (f) representative curves. PVA/BA adhesive films were activated with water for 10 seconds and adhered to substrate at 37° C. for 2 minutes.

FIG. 5. Rheological response of PVA/BA solution: the stress response of (a) PVA/BA 5/1 and (b) 15/1 solution (20 w % in DI water) during shear sweep; the oscillation stress of (c) PVA/BA 5/1 and (d) 15/1 solution (20 w % in DI water) in frequency sweep (filled square: oscillation stress with increasing frequency; empty square: oscillation stress with decreasing frequency).

FIG. 6. Mechanical measurements of PVA/BA films: representative (a) stress-strain curves of PVA/BA 5/1, 15/1, 30/1, and 50/1 films with (b) good repeatability (PVA/BA 5/1 three repeated stress-strain curves shown).

FIG. 7. Optimization and reversibility of PVA/BA adhesion on glasses: the influence of (a) film thickness (10-second hydration time), (b) hydration time (0.12 mm thick film), and (c) adhesion evolution (10-second hydration time, 0.12 mm thick film) of PVA/BA 15/1 films in lap shear tests; the (d) adhesion comparison before and after 30-second water submission for PVA/BA 15/1 films in tensile tests.

FIG. 8. Photo demonstration of (a) adhesive failure from highly crosslinked PVA/BA films (5/1 and 15/1) and (b) cohesive failure from loosely crosslinked PVA/BA films (30/1 and 50/1).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Adhesion bonding at interfaces falls into two categories: 1) strong but slow and irreversible covalent bonds and 2) fast, reversible but weak non-covalent bonds. Synergizing the advantages from both categories remains challenging but pivotal for next-generation wound adhesives. Here we reported the first example utilizing dynamic covalent bonds as wound dressing adhesives to achieve fast and reversible adhesion with strong adhesion outperforming commercial superglue product. In an example, non-limiting illustration, the boronic ester bonds in polyvinyl alcohol (PVA) and boric acid (BA) network provide 61 N/cm² shear adhesion and 511 N/cm² transcutaneous adhesion within 2 minutes and allows effortless debonding with excess water.

The mechanical properties of PVA/BA adhesives are tunable through crosslinking density. Upon water activation (within seconds), the surface boronic ester bonds in PVA/BA film undergo fast debonding and instant softening, thus maximizing conformal contact with the adherends and reforming boronic ester bonds at the interfaces. Meanwhile, the bulk films remain dehydrated guaranteeing efficient load transmission to afford strong adhesion. On surfaces with (hairy mouse skins) or without (glass) functional groups, PVA/BA adhesive demonstrate superior adhesion comparing to commercial wound adhesive superglue (Dermabond®).

Adsorption adhesion theory reveals the importance of interfacial binding between adhesives and adherends. Covalent bonds represent the strongest interactions (150-950 kJ/mol) and find most applications in superglue adhesives which has strong adhesion but requires long adhering time (hours to days) or external stimuli (UV, heat, etc.) with no facile reversibility. On the contrary, weaker non-covalent interactions, such as van der Waals forces (2-15 kJ/mol) and hydrogen bonds (10-40 kJ/mol), demands shorter adhering time (seconds to minutes) with reversibility but weaker adhesion.

Naturally, two adhesion property trade-offs arise: 1) adhesion strength vs. reversibility; 2) adhesion strength vs. adhesion time. Especially for wound adhesives, solving both trade-offs are ideal for both patients and doctors. Currently wound closure relies either on sutures (or staples) or wound dressing adhesives. While sutures provide strong force for wound closure, they are also time-consuming, painful to patients, and vulnerable to infections. Wound dressing adhesives, on the other hand, are soft, non-invasive, but normally offer low adhesion force.

Various attempts have been exploited to tackle the two trade-offs. Surfaces with nano- or micro-topological designs inspired from nature (such as geckos) achieved adhesion (<10 N/cm²) with excellent reversibility by accumulating non-covalent interactions; however, the adhesion strength is proportional to the complicity and density of the topological elements which requires time-consuming and expensive manufacturing. We reported a hydrogel-based adhesive to realize superglue adhesion (892 N/cm²) and facile reversibility through non-covalent interactions from shape adaption mechanism. But the adhesion and reverse process require hours to complete, which is detrimental for applications like wound addressing. Commercial product, such as Dermabond©, accelerate the adhering process to less than a few minutes through a two-part design. Due to its cyanoacrylate chemistry Dermabond® can lose reversibility. To truly overcome the two trade-offs at the same time, a middle ground between covalent bonds and non-covalent interactions seems inevitable.

Dynamic covalent bonds, covalent bonds that are capable of exchanging bond connectivity between multiple molecules, synergize the strong covalent bond energy and the dynamic bond exchange (similar to non-covalent bonds, such as hydrogen bonds). Commonly studied reactions with dynamic covalent bonds include transesterification, nucleophilic substitution, imine chemistry, Diels-Alder reaction, disulfide exchange, thiol-X chemistry, boronic esters, and silyl ethers. Most dynamic covalent bonds require either high temperatures, long reaction time (hours), catalysts/initiators, or external stimuli.

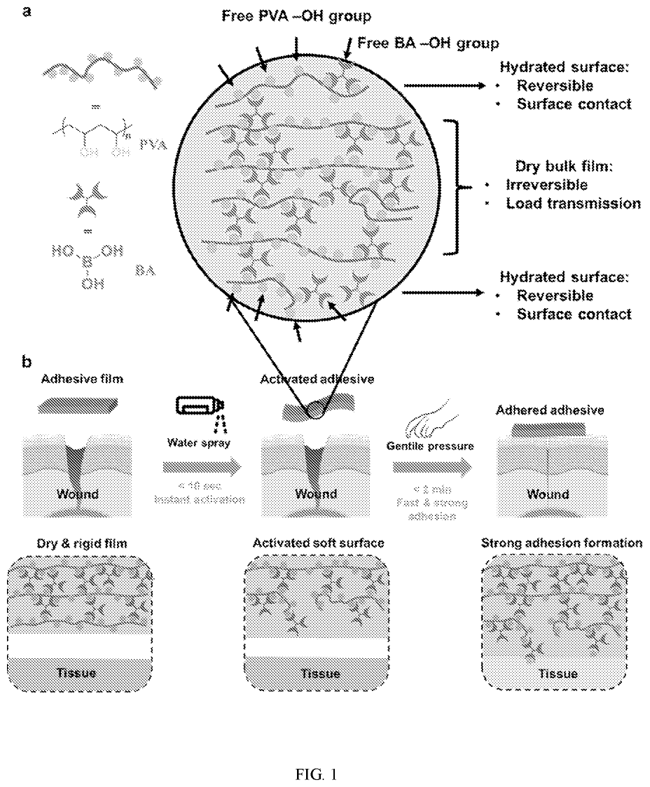

The unique properties of boronic esters allow us to create adhesives basing on diol-containing polymers crosslinked by boric acids or boronic acids. For wound adhesives, biocompatible and water-soluble polyvinyl alcohol (PVA) and boric acid (BA) are chosen. BA reacts with the hydroxyl groups of PVA chains to create crosslinked materials which is stable at alkaline pH (higher than the pKa of BA 9.2) or soluble in water at neutral pH. As an illustration of the disclosed technology, we solution-cast PVA/BA water solution into dry films to eliminate water evaporation process completely. The dry PVA/BA film is activated by water within seconds leading to a hydrated surface (dynamic nature) and a dry bulk inner film (covalent nature) (FIG. 1a). The hydrated surface undergoes instant debonding as neutral pH favors the debonding of boronic ester bonds. The debonding softens the surface, allows good contact between the PVA/BA film and adherends, and reforms covalent bonds with the adherends as the activation water evaporates (FIG. 1b). The dry bulk film remains strong, provides film integrity, and dissipates force efficiently.

### Example Non-Limiting Results and Discussion

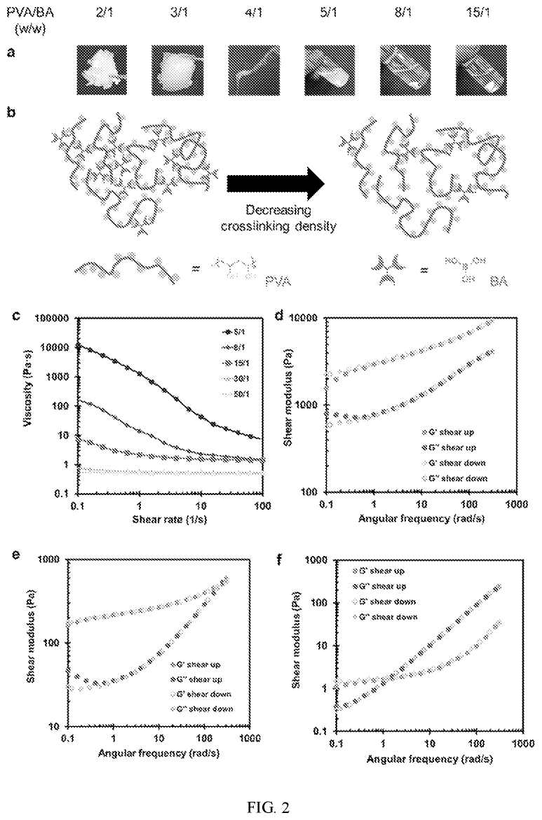

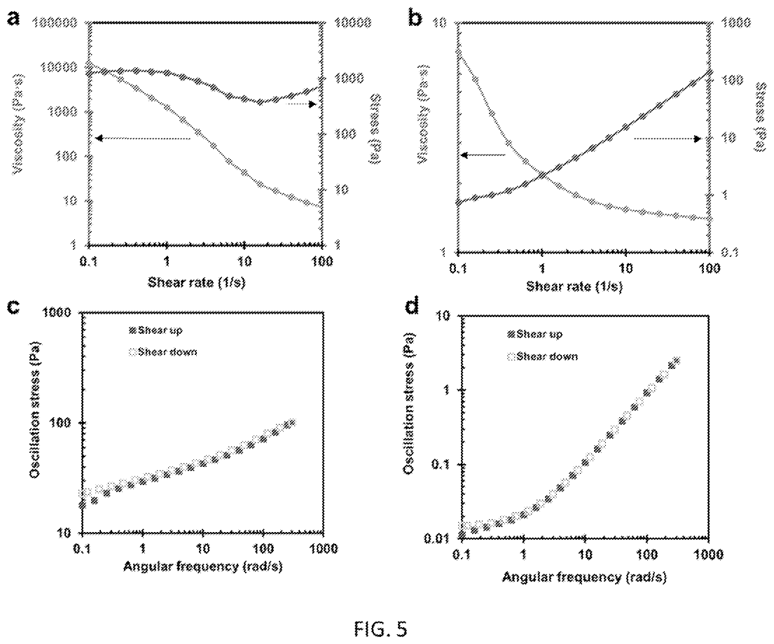

Rheological behavior of PVA/BA solution. At neutral pH 7, addition of boric acid (BA) into polyvinyl alcohol (PVA) solution led to instant white gel precipitates (Movie Si). FIG. 2a depicted different physical appearances of PVA/BA gels/solution generated in neutral water at 20 w %. High crosslinking densities (PVA/BA 2/1 and 3/1) produced elastic materials while PVA/BA 5/1, 8/1, and 15/1 gels redissolved completely into homogenous solution (FIG. 2b). PVA/BA X/Y refers to the weight ratio between PVA (X) and BA (Y) while the combined concentration of PVA+BA is kept at a constant level (20 w %). For homogenous PVA/BA solution, we observed a steady viscosity decrease with decreasing crosslinking densities (from PVA/BA 5/1 to 50/1, FIG. 2c) during a shear sweep process. All solutions demonstrated shear thinning as expected for polymer solution due to chain alignments at high shear rates. The less crosslinked solution (PVA/BA 15/1) exhibited linear stress-shear rate response similar to ideal materials, while the more crosslinked solution (PVA/BA 5/1) demonstrated non-linear stress response at high shear rate as non-ideal materials (FIGS. 5a and 5b). To demonstrate the dynamic nature of the boronic ester bonds, frequency sweep of PVA/BA solution was conducted. The shear storage modulus (G′) remained higher than the shear loss modulus (G″) in PVA/BA 5/1 solution, indicating a solid-like behavior throughout the entire frequency range tested (FIG. 2d). Although PVA/BA 5/1 solution flows easily at ambient condition, the dynamic crosslinks (boronic ester bonds) presented a solid-like stress response. Another important observation is that, with increasing frequency, the discrepancy between G′ and G″ diminished which is counter-intuitive for most polymer solution or polymer melt. At high frequency, most polymer solutions undergo incomplete relaxation due to the short time span. As a result, the materials behave more like a solid, hence a large discrepancy between G′ and G″. Interestingly, in the PVA/BA 5/1 example, a reverse trend was observed. Further, in PVA/BA 8/1 (FIG. 2e) and PVA/BA 15/1 (FIG. 2f) solutions, this trend continuously grew, and a cross-over point of G′ and G″ appeared symbolizing a transition from solid-like behavior at low frequency to liquid-like behavior at high frequency. The rationale of this rare trend hides behind the increasing oscillation stress with increasing frequency. FIG. 5c and FIG. 5d revealed two orders of magnitude increase of oscillation stress with increasing frequency. The increased oscillation stress ruptured boronic ester bonds, and the weakened materials deformed readily, just like a liquid. Comparing the modulus value with increasing and decreasing frequency (blue and red symbols in FIG. 2d, 2e, and 2f), the water evaporation is negligible within the experiment time as both values overlapped with each other.

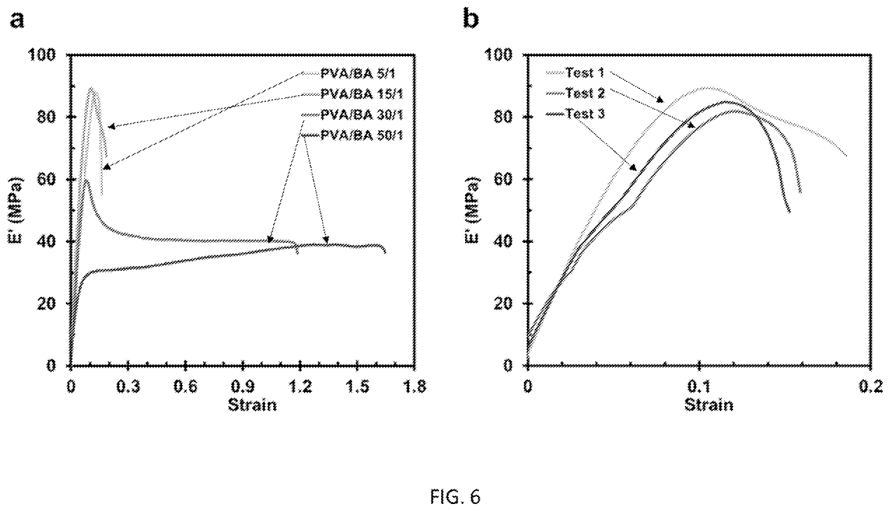

Adhesion study on model substrates. To dramatically shorten the develop time of the PVA/BA adhesives, we solvent cast the PVA/BA solution (20 w % in water) into dry films. Rehydration of the PVA/BA dry films will instantly soften the surfaces for adhesion while the dry inner film provides high modulus for load transition (FIG. 1). As crosslinking density decreased, the modulus of dry film decreased as expected (FIG. 6a) with good repeatability (FIG. 6b). Table 1 summaries the mechanical properties of PVA/BA dry films: decreased crosslinking density leads to lower Young's modulus and ultimate modulus but longer strain at break and higher toughness.

TABLE 1
Summary of PVA/BA film mechanical properties. All
values are reported as the mean values (from at
least three repeats) with standard deviations.

| | Young's | Ultimate | | |
|---|---|---|---|---|
| | modulus | modulus | Strain | Toughness |
| Composition | (MPa) | (MPa) | at break | (MJ/m³) |
| PVA/BA 5/1 | 913 ± 24 | 89.0 ± 5.3 | 0.15 ± 0.02 | 8.52 ± 1.84 |
| PVA/BA 15/1 | 1160 ± 139 | 85.3 ± 3.7 | 0.17 ± 0.02 | 10.23 ± 1.94 |
| PVA/BA 30/1 | 761 ± 177 | 52.0 ± 7.4 | 1.29 ± 0.35 | 45.36 ± 8.77 |
| PVA/BA 50/1 | 517 ± 147 | 33.1 ± 5.1 | 1.89 ± 0.25 | 54.16 ± 3.19 |

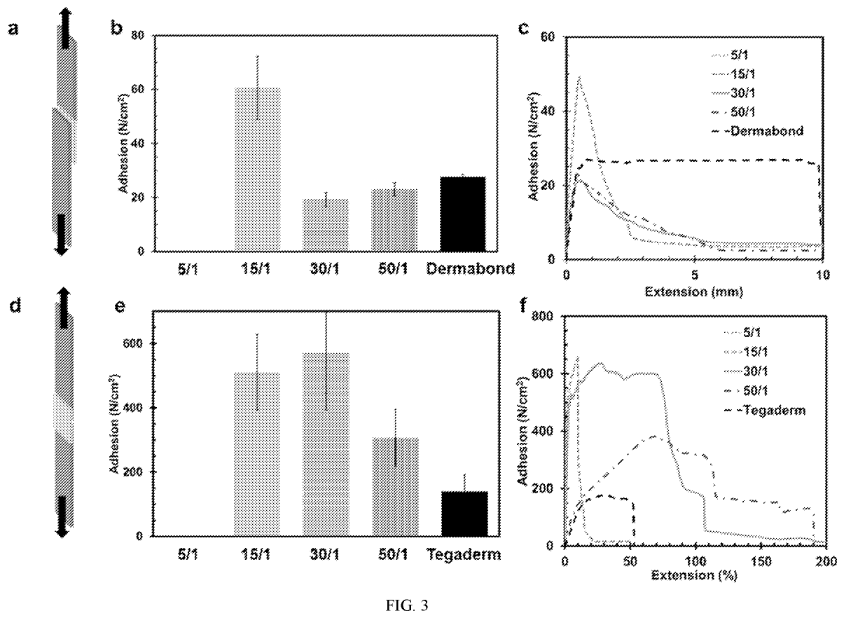

We firstly tested the adhesion of PVA/BA films on glass. After hydration in water for 10 seconds, PVA/BA films with various compositions demonstrated strong shear and transcutaneous adhesion comparing to commercial wound adhesive products after 2 minutes of adhering time. In lap shear tests (FIG. 3a), PVA/BA 15/1 demonstrated the highest shear adhesion at 60.6±11.7 N/cm² (FIG. 3b) after 2 minutes adhering time, doubling the performance of commercial wound adhesive Dermabond® (27.6±0.9 N/cm²). Further, PVA/BA adhesives are reversible after exposure to water for 30 seconds (discussed in the tensile test section later) while Dermabond® is irreversible. Less crosslinked PVA/BA 30/1 (19.1±2.7 N/cm²) and 50/1 (23.0±2.3 N/cm²) exhibited similar adhesion to Dermabond®. In contrast, more rigid PVA/BA 5/1 showed essentially no adhesion as 10 seconds are too short to soften its highly crosslinked surfaces. Previously reported adhesion was less than 10 N/cm², within minutes. In the representative adhesion stress-displacement curve (FIG. 3c), all PVA/BA adhesives showed a typical peak adhesion at the early stage of the experiments with cohesive failures. However, as a liquid adhesive, Dermabond® was not fully solidified after 2 minutes leading to a plateaued adhesion value for the initial 10 mm.

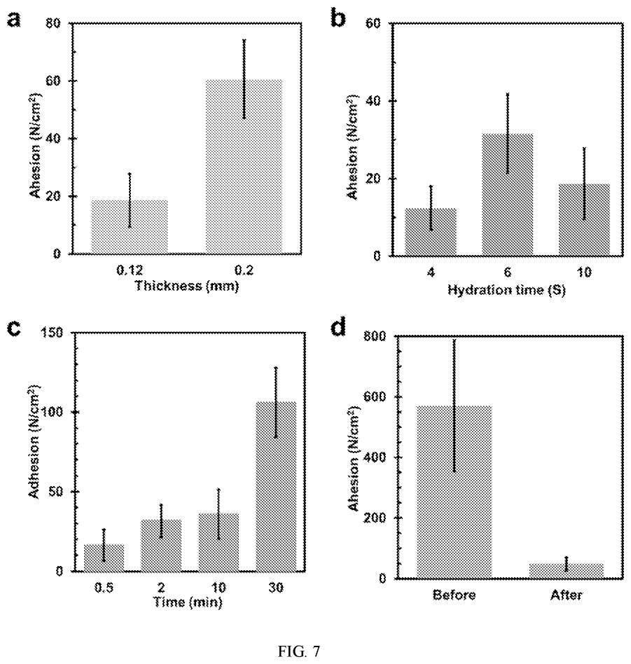

Without being bound to any particular theory, the adhesion preparation procedure can be sensitive to film thickness, hydration time, and adhering time. Three times adhesion improvement (FIG. 7a) was achieved after increasing the PVA/BA 15/1 thickness from 0.12 mm (18.7±9.1 N/cm²) to 0.2 mm (60.6±11.7 N/cm²). Similarly, for PVA/BA 15/1 film with 0.12 mm thickness, the optimized hydration time is 6 seconds (31.6±10.2 N/cm², FIG. 7b). Shorter hydration time (4 seconds, 12.4±5.6 N/cm²) led to insufficient softening on the surfaces while longer hydration time (10 seconds, 18.7±9.1 N/cm²) deteriorated film integrity. Even PVA/BA 5/1 can be properly activated after 1 minute's hydration. Increasing adhering time can also alter the shear adhesion significantly. Shear adhesion after 30 minutes adhering time reached 106±22 N/cm² in comparison to 16.4±9.8 N/cm² after 30 seconds (FIG. 7c).

Another aspect for wound adhesives is the transcutaneous adhesion, the force provided by the adhesives to close the wound. We employed a tensile test to test the transcutaneous adhesion: a PVA/BA adhesive film was adhered to two glass slides after which uniaxial force was applied on both glass slides (FIG. 3d). FIG. 3e revealed that PVA/BA 15/1 and 30/1 yielded the highest adhesion at 510±117 N/cm² and 570±177 N/cm² respectively, which is orders of magnitude higher than reported wound adhesives even after hours or days of development. But due to their differences in crosslinking density, PVA/BA 30/1 showed significantly longer extension before break (>200%) than PVA/BA 15/1 (˜50%, FIG. 3f). PVA/BA 50/1 showed lower transcutaneous adhesion at 306±89 N/cm² which is still significantly higher than previous wound adhesion literatures or commercial products (Tegaderm®).

In the transcutaneous adhesion tests, the adhesion performance relies both on shear adhesion as well as the film modulus. Thus, low modulus (PVA/BA 50/1) is detrimental to the transcutaneous performance. We observed no adhesion from PVA/BA 5/1 as its shear adhesion is negligible. FIG. 7d exhibited the easy removal of PVA/BA adhesive films. After 30 seconds of submersion in water, the transcutaneous adhesion dropped from 570±177 N/cm² to 49±23 N/cm². PVA/BA films completely dissolved in water after 2 hours.

### Adhesion Tests on Mouse Skins

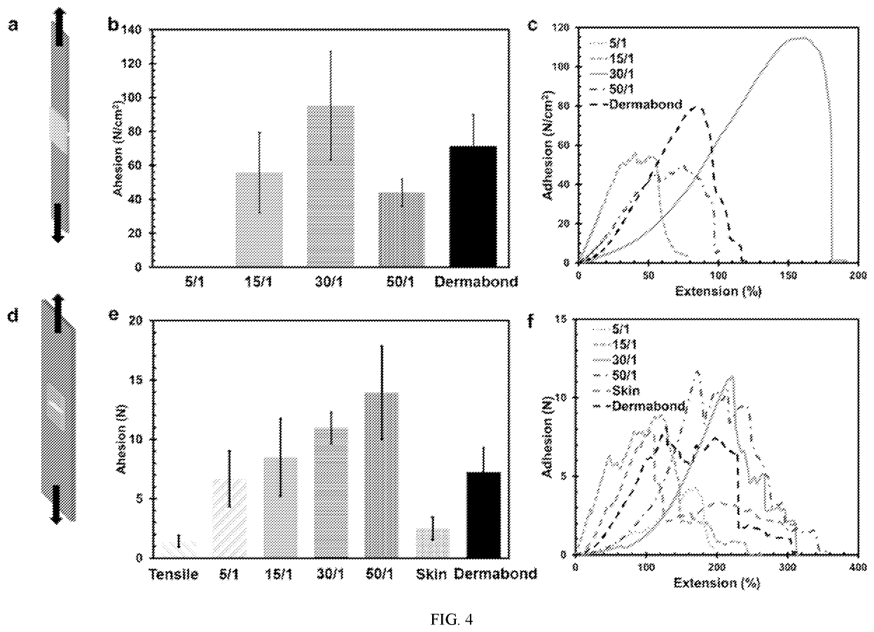

Glass is a non-functional substrate with minimal surface complexity; however, wound adhesives often encountered complicated surfaces (such as hair). To quantify the adhesion performance of PVA/BA adhesive films as wound adhesives, we employed hairy mouse skins as adherends. PVA/BA adhered to mouse skins with both higher transcutaneous (FIG. 4a) and incision (FIG. 4d) adhesion comparing to commercial product Dermabond®. Due to the complexity of mouse skins, both PVA/BA and Dermabond® recorded lower transcutaneous adhesions comparing to those on glasses. Nevertheless, PVA/BA 30/1 still demonstrated strong adhesion of 95±32 N/cm² (FIG. 4b) after 2 minutes when most wound adhesives reported less than 30 N/cm² adhesion after hours of adhering.² Dermabond© showed a slightly lower adhesion of 71.3±18.6 N/cm² ascribing to its liquid nature which overcomes hairs on mouse skins easier. PVA/BA 15/1 and 50/1 also showed promising adhesion at 55.8±23.6 N/cm² and 43.9±8.0 N/cm² respectively. PVA/BA 5/1 stayed inactivated after 10 seconds hydration time. In the representative stress-strain curves (FIG. 4c), PVA/BA 30/1 provided the longest extension due to its optimized crosslinking density. The higher modulus of PVA/BA 15/1 creates a modulus mismatch between the adhesive films and the soft mouse skins and decreases the shear adhesion at the interfaces. The less crosslinked PVA/BA 50/1 guarantees a good shear adhesion, but its inferior tensile properties lead to a lower transcutaneous adhesion.

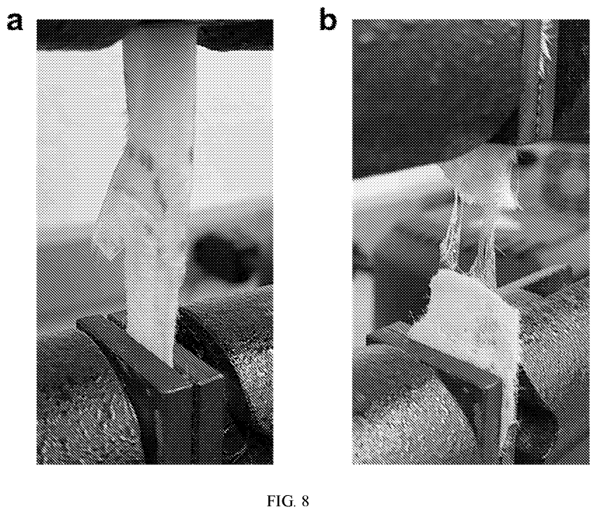

For most wound treatments, the skins around the wound site remained connected. Hence, to better mimic the actual wound site condition, we employed an incision test (FIG. 4d) to test PVA/BA's adhesion on hairy mouse skins. Due to its liquid nature, it is hard to control Dermabond® thickness in this experiment. Instead, FIG. 4e and FIG. 4f demonstrated the peak force instead of peak pressure as a function of extension. As the skin contributes partially to the tensile properties, a good contact at the interfaces between adhesives and adherends is pivotal to generate strong adhesion. PVA/BA 50/1 has the lowest crosslinking density and thus lowest modulus to provide the best contact to the skins. As a result, PVA/BA 50/1 exhibited the strongest adhesion (FIG. 4e) at 13.9±3.9 N (or 1159 327 N/cm²) which is roughly twice of the Dermabond® adhesion (7.3±2.0 N). With increasing crosslinking density, the adhesion decreased from 11.0±1.3 N (PVA/BA 30/1) to 6.7±2.3 N (PVA/BA 5/1). PVA/BA incision tests demonstrated synergistic adhesion as the accumulated adhesion of the control experiments on bare skins (with incision but no adhesives) and tensile tests fell short to PVA/BA adhesives (FIG. 4e). FIG. 4f depicts representative force-strain curves during the incision tests. The peak adhesion of high-modulus adhesives (PVA/BA 5/1, 15/1 and Dermabond®) coincided with bare skin control (at ˜100% strain) as their poor interfacial contact led to adhesive failures (FIG. 8a). Conversely, softer adhesives (PVA/BA 30/1 and 50/1) benefited from better contact to hairy skins and peaked at longer extension (>²⁰⁰% strain) with cohesive failures (FIG. 8b).

In summary, we successful introduced dynamic covalent bonds into wound adhesives to achieve fast and reversible adhesion which outperforms commercial super-glue based products. Partial hydration of the PVA/BA films allows fast debonding and thus softening at the surfaces, allowing good contact and instant adhering to complicated substrates. At the same time, the dry inner film retains high Young's modulus (500-1000 MPa) to transmit load efficiently. Synergistically, the PVA/BA films demonstrated superior transcutaneous adhesion on both glass (570±177 N/cm²) and mouse skins (95±32 N/cm²) comparing to commercial wound adhesives. PVA/BA adhesives achieves orders of magnitude stronger and faster adhesion with effortless removal compared to previously reported wound adhesives.

### Materials and Methods

### Materials.

Poly(vinyl alcohol) (Mw 13,000-23,000 g/mol, 87-89% hydrolyzed) was purchased from Sigma-Aldrich and used without further treatment. Boric acid (DNase, RNase and Protease free, 99.5%) was purchased from Acros Organics and used directly. Mouse skin.

### PVA/BA Solution

PVA was dissolved in deionized water first to form a homogeneous solution. With vigorous stirring, BA solids were charged into the solution directly. Instant white precipitates appeared. Continuous stirring redissolved the precipitates to yield a homogeneous solution again at room temperature. The combined weight percent of PVA and BA was kept at constant (20%) while the relative ratio between PVA and BA was varied. Take the preparation of PVA/BA 15/1 solution as an example: 1) PVA (2.34 g) was dissolved in deionized water (10 g) overnight; 2) BA (0.16 g) was added into the PVA solution directly with vigorous stir, and instant white precipitates formed; (3) Continue stirring (24 hours) redissolved the white precipitates and formed homogenous solution.

### PVA/BA Film Preparation

The PVA/BA aqueous solution (20 w %) was casted on glass substrates. Doctor blades were utilized to maintain a uniform solution thickness (2 mm). The casted solution was left drying at ambient condition for 48 hours with glass cover to prevent dust. The dried films were peeled off from the glass substrates directly to afford free-standing films.

### Rheological Measurements

All rheological measurements were conducted on a Discovery Hybrid Rheometer HR 20 with 40 mm 0.9983330 cone plate (UHP Steel). The gap was kept at 1000 mm, and the temperature was maintained at 25° C.

The shear sweep was performed from 0.1 to 100 S⁻¹. The strain sweep was conducted from 0.01 to 50% at 1 Hz. The frequency sweep was conducted from 0.1 to 300 rad/s first and 300 to 0.1 rad/s second at 1% strain.

### Adhesion Tests

All adhesion tests were performed on an Instron (5564 Tabletop Materials Testing System) machine in tensile mode at 5 mm/min rate and room temperature. Each test is repeated at least three times and reported as the average value with standard deviation.

### Sample Preparation

PVA/BA films were cut into rectangular shape (10×10×0.02 mm) before hydration in deionized water for 10 seconds. For lap shear test, both sides were hydrated; while only one side was wet for tensile and incision tests. The activated adhesives were adhered to untreated substrates (glass or mouse skins) with 200 g weight on top to guarantee good contact. The entire sample was placed on hot plate (37° C. to mimic body temperature) and developed for 2 minutes before adhesion tests. As liquid adhesives, Scotch tapes were used to confine the adhesive areas of Dermabond®. In incision tests, the mouse skins were cut into 2×4 cm rectangular shape followed by a 1 cm incision at the middle the skins.

For the reversibility tests (easy removal), the tensile sample preparation was identical as described above. After 2 minutes of adhering time, the tensile samples were submerged into deionized water for additional 30 seconds before subjecting to adhesion tests.

Data report. The adhesion in lap shear tests were calculated using peak force (divided by length×width area). In tensile tests, the adhesion was reported as the ratio of peak force and film cross-section area (width×thickness). In incision tests, peak force was directly utilized to represent adhesion.

### Aspects

The following Aspects are illustrative only and do not limit the scope of the present disclosure or the appended claims. Any part or parts of any one or more Aspects can be combined with any part or parts of any one or more other Aspects.

Aspect 1. A reversibly adhesive film, comprising: at least two hydroxyl-bearing polymer chains, the at least two hydroxyl-bearing polymer chains being crosslinked by crosslinks that comprise one or more boronic ester bonds, the film having accessible hydroxyl groups at a surface of the film, and at least a portion of the film being in a dried state.

Aspect 2. The film of Aspect 1, wherein the boronic ester bonds are derived from reaction between a boric acid or a boronic acid and a hydroxyl of a polymer chain.

Aspect 3. The film of any one of Aspects 1-2, further comprising an amount of boric acid, an amount of a boronic acid, or both.

Aspect 4. The film of Aspect 3, wherein the boronic acid is one or more of phenylboronic acid, a phenylboronic acid derivative, a diboronic acid, a multiboronic acid, an aromatic boronic acid with a substitution, or any combination thereof. Phenylboronic acids and their derivatives are considered especially suitable. Without being bound to any particular theory or embodiment, the use of a phenylboronic acid can slow the debonding process (of the boronic ester bonds) when the adhesive film is exposed to water.

Aspect 5. The film of any one of Aspects 1-4, wherein a polymer chain comprises a diol. Polymer chains that include two (or more) hydroxyls on a repeat unit are considered suitable, e.g., polyols.

Aspect 6. The film of Aspect 5, wherein polymer chains comprise polyvinyl alcohol (PVA).

Aspect 7. The film of any one of Aspects 1-6, wherein the film comprises (i) a hydrated surface and (ii) an interior, the hydrated surface optionally having a thickness in the range of from about 100 nm to about 100 μm, and the interior optionally having a thickness in the range of from about 100 μm to about 1 cm.

A hydrated surface can be created by, e.g., exposing the surface of the film to water, e.g., by spraying, dipping, or otherwise contacting the surface of the film to water.

The hydrated surface can have a thickness in the range of from about 100 nm to about 100 μm, or from about 200 nm to about 50 μm, or from about 300 nm to about 10 μm, and all intermediate values and ranges.

The interior of the film (which can remain dry while the film surface is hydrated) can have a thickness of from about 100 μm to about 1 cm, or from about 200 μm to about 0.5 cm, or from about 300 μm to about 0.1 cm (i.e., 1 mm), and all intermediate values and ranges.

Without being bound to any particular theory or embodiment, a hydrated surface of the adhesive film may exhibit a Young's modulus that differs from the Young's modulus of the interior of the adhesive film, thereby allowing the surface of the film to conform well to an adherend to which the adhesive film is applied while the interior of the film remains relatively stiff, thereby providing some structural support to the film and facilitating handling and placement of the film by a user.

Aspect 8. The film of Aspect 7, wherein the hydrated surface of the film comprises a Young's modulus in the range of from about 100 Pa to about 10 MPa, e.g., from about 100 Pa to about 10 MPa, from about 200 Pa to about 5 MPa, from about 500 Pa to about 1 MPa, from about 1 kPa to about 1 MPa, from about 10 kPa to about 1 MPa, from about 100 kPa to about 1 MPa, and all intermediate values and ranges.

Aspect 9. The film of any one of Aspects 7-8, wherein the interior of the film comprises a Young's modulus in the range of from about 100 MPa to about 5000 MPa, e.g., from about 100 MPa to about 5000 MPa, from about 250 MPa to about 2500 MPa, from about 500 MPa to about 1000 MPa, and all intermediate values and ranges.

Aspect 10. The film of any one of Aspects 1-9, wherein the film defines a Young's modulus, when dry, in the range of from about 100 MPa to about 5000 MPa, e.g., from about 100 MPa to about 5000 MPa, from about 250 MPa to about 2500 MPa, from about 500 MPa to about 1000 MPa, and all intermediate values and ranges.

Aspect 11. The film of any one of Aspects 1-10, wherein the surface of the film exhibits a reversible single-joint lap shear adhesion on glass in the range of from about 10 N/cm² to about 70 N/cm² (e.g., from about 10 to about 70 N/cm², from about 15 to about 65 N/cm², from about 20 to about 60 N/cm², from about 25 to about 55 N/cm², from about 30 to about 50 N/cm², from about 35 to about 45 cm², or about 40 N/cm²) upon 2 minutes of contact with the glass following 10 seconds of surface hydration.

Aspect 12. The film of any one of Aspects 1-11, wherein the surface of the film exhibits a reversible tensile adhesion on glass with peak adhesion in the range of from about 100 N/cm² to about 600 N/cm² (e.g., from about 100 to about 600 N/cm², from about 150 to about 550 N/cm², from about 200 to about 500 N/cm², from about 250 to about 450 N/cm², from about 300 to about 400 N/cm², or about 350 N/cm²) upon 2 minutes of contact with the glass following 10 seconds of surface hydration.

Aspect 13. The film of any one of Aspects 1-12, further comprising a water-impervious packaging within which the film is disposed. Such a packaging can be, e.g., configured as a bandage package, which bandage package can be removed by the user at the time of adhesive film use, e.g., in a medical care setting or even in a field setting.

Aspect 14. The film of any one of Aspects 1-13, wherein the film is derived from a composition that comprises (i) the hydroxyl-bearing polymer and (ii) one or both of boric acid and boronic acid, the weight ratio of (i) to (ii) in the composition optionally being from about 50:1 to 3:1, e.g., from about 50:1 to about 3:1, from about 45:1 to about 3.5:1, from about 40:1 to about 5:1, from about 35:1 to about 4:1, from about 30:1 to about 5:1, from about 35:1 to about 6:1, from about 30:1 to about 8:1, from about 25:1 to about 10:1, from about 20:1 to about 15:1, or even about 17:1.

Aspect 15. The film of Aspect 14, wherein the combined weight of (i) and (ii) in the composition is up to about 30 wt % of the composition, more preferably up to about 20 wt % of the composition. Combined weights of (i) and (ii) can be, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or even about 30% of the weight of the composition.

Aspect 16. The film of any one of Aspects 1-15, wherein the film, when adhered to skin, can be manually peeled off following 30 seconds of hydration.

Aspect 17. The film of any one of Aspects 1-16, wherein the film defines a thickness of from about 100 μm to about 1 cm, e.g., from about 100 μm to about 1 cm, from about 250 μm to about 750 μm, or about 500 μm.

Aspect 18. A method, comprising contacting a hydrated portion of a film according to any one of Aspects 1-17 to an adherend for a time sufficient to give rise to adhesion between the film and the adherend.

Aspect 19. The method of Aspect 18, wherein the adherend is a tissue. The disclosed films can be applied to any part of the body, e.g., skin, vascular tissue, oral tissue (e.g., gums, cheek interior), nasal tissue, cardiac tissue, gastrointestinal tissue, and the like.

Aspect 20. The method of Aspect 19, wherein the tissue is skin tissue, oral tissue, vascular tissue, or any combination thereof.

Aspect 21. A method, comprising hydrating a film according to any one of Aspects 1-17 that is adhered to an adherend so as to reduce adhesion between the film and the adherend.

Aspect 22. A reversible adhesive, comprising: a first film layer, the first film layer comprising at least two first hydroxyl-bearing polymer chains crosslinked by first crosslinks that comprise one or more boronic ester bonds; and a second film layer, the second film layer comprising at least two second hydroxyl-bearing polymer chains crosslinked by second crosslinks that comprise one or more boronic ester bonds, wherein (1) the first hydroxyl-bearing polymer chains differ from the second hydroxyl-bearing polymer chains in one or more of composition and concentration, or (2) the boronic ester bonds are present in the first film layer at a different density than the boronic ester bonds in the second film layer, or (3) both (1) and (2).

In this way, one can form a multi-layer adhesive film. This can present the advantage of different films being suitable for different applications. For example, a user may desire a film having a backing layer that has a certain Young's modulus that is well-suited for film handling and placement, while having differently-configured patient-contacting layers so that one type of multi-layer adhesive film has a comparatively soft and conformable patient-facing layer that is well-suited for use on soft infant skin, while another type of multi-layer adhesive film has the same backing layer but has a patient-facing layer that is less soft than a layer used with infants. The disclosed multi-layer adhesives can be hydrated to prepare them for adhesion, and can also be wetted—after adhered—to release their adhesion.

Aspect 23. The reversible adhesive of Aspect 22, wherein at least one of the first hydroxyl-bearing polymer chains and the second hydroxyl-bearing polymer chains is polyvinyl alcohol (PVA).

Aspect 24. The reversible adhesive of any one of Aspects 22-23, wherein the boronic ester bonds of the first film and/or the boronic ester bonds of the second film comprise a phenyl. As described elsewhere herein, the use of phenylboronic acid can be beneficial in certain applications.

Aspect 25. The reversible adhesive of any one of Aspects 22-24, wherein the first film layer and the second film layer differ in Young's modulus when dry.

Aspect 26. The reversible adhesive of any one of Aspects 22-25, wherein the first film layer and the second film layer exhibit different adhesive strengths when hydrated.

1. A reversibly adhesive film, comprising:
at least two hydroxyl-bearing polymer chains,
the at least two hydroxyl-bearing polymer chains being crosslinked by crosslinks that comprise one or more boronic ester bonds,
the film having accessible hydroxyl groups at a surface of the film, and
at least a portion of the film being in a dried state,
the film optionally defining a thickness of from about 100 μm to about 1 cm.

2. The film of claim 1, wherein the boronic ester bonds are derived from reaction between a boric acid or a boronic acid and a hydroxyl of a polymer chain.

3. The film of claim 1, further comprising an amount of boric acid, an amount of a boronic acid, or both.

4. The film of claim 3, wherein the boronic acid is one or more of phenylboronic acid, a phenylboronic acid derivative, a diboronic acid, a multiboronic acid, an aromatic boronic acid with a substitution, or any combination thereof.

5. The film of claim 1, wherein a polymer chain comprises a diol.

6. The film of claim 5, wherein polymer chains comprise polyvinyl alcohol (PVA).

7. The film of claim 1, wherein the film comprises (i) a hydrated surface and (ii) an interior, the hydrated surface optionally having a thickness in a range of from about 100 nm to about 100 μm, and the interior optionally having a thickness in the range of from about 100 μm to about 1 cm.

8. The film of claim 7, wherein (a) the hydrated surface of the film comprises a Young's modulus in the range of from about 100 Pa to about 10 MPa, (b) the interior of the film comprises a Young's modulus in the range of from about 100 to about 5000 MPa, or both (a) and (b).

9. (canceled)

10. The film of claim 1, wherein (a) the film defines a Young's modulus, when dry, in the range of from about 100 to about 5000 MPa, (b) the surface of the film exhibits a reversible single-joint lap shear adhesion on glass in the range of from about 10 to about 70 N/cm2 upon 2 minutes of contact with the glass following 10 seconds of surface hydration, or both (a) and (b).

11. (canceled)

12. The film of claim 1, wherein the surface of the film exhibits a reversible tensile adhesion on a glass with peak adhesion in the range of from about 100 to about 600 N/cm² upon 2 minutes of contact with the glass following 10 seconds of surface hydration.

13. The film of claim 1, further comprising a water-impervious packaging within which the film is disposed.

14. The film of claim 1, wherein the film is derived from a composition that comprises (i) the hydroxyl-bearing polymer and (ii) one or both of boric acid and boronic acid, a weight ratio of (i) to (ii) in the composition optionally being from about 50:1 to 3:1.

15. The film of claim 14, wherein the combined weight of (i) and (ii) in the composition is up to about 30 wt % of the composition, more preferably up to about 20 wt % of the composition.

16. The film of claim 1, wherein the film, when adhered to skin, can be manually peeled off following 30 seconds of hydration.

17. (canceled)

18. A method, comprising contacting a hydrated portion of a film according to claim 1 to an adherend for a time sufficient to give rise to adhesion between the film and the adherend, the adherend optionally being a tissue.

19. (canceled)

20. The method of claim 18, wherein the adherend is a tissue and wherein the tissue is skin tissue, oral tissue, vascular tissue, or any combination thereof.

21. A method, comprising hydrating a film according to claim 1 that is adhered to an adherend so as to reduce adhesion between the film and the adherend.

22. A reversible adhesive, comprising:
a first film layer,
the first film layer comprising at least two first hydroxyl-bearing polymer chains crosslinked by first crosslinks that comprise one or more boronic ester bonds; and
a second film layer,
the second film layer comprising at least two second hydroxyl-bearing polymer chains crosslinked by second crosslinks that comprise one or more boronic ester bonds,
wherein
(1) the first hydroxyl-bearing polymer chains differ from the second hydroxyl-bearing polymer chains in one or more of composition and concentration, or
(2) the boronic ester bonds are present in the first film layer at a different density than the boronic ester bonds in the second film layer, or
(3) both (1) and (2).

23. The reversible adhesive of claim 22, wherein (a at least one of the first hydroxyl-bearing polymer chains and the second hydroxyl-bearing polymer chains is polyvinyl alcohol (PVA), (b) the boronic ester bonds of the first film and/or the boronic ester bonds of the second film comprise a phenyl, or both (a) and (b).

24. (canceled)

25. The reversible adhesive of 22, wherein (a) the first film layer and the second film layer differ in Young's modulus when dry, (b) first film layer and the second film layer exhibit different adhesive strengths when hydrated, or both (a) and (b).

26. (canceled)
